(19) United States
(12) Patent Application Publication
(10) Pub. No.: US 2026/0125381 A1
(43) Pub. Date: May 7, 2026

(54) NOVEL Kv3 CHANNEL MODULATORS

(71) Applicant: Merck Sharp & Dohme LLC, Rahway, NJ (US)

(72) Inventors: Anthony T. Ginnetti, Perkasie, PA (US); Shawn J. Stachel, Perkasie, PA (US); Linda M. Suen-Lai, Malvern, PA (US)

(73) Assignee: Merck Sharp & Dohme LLC, Rahway, NJ (US)

(21) Appl. No.: 19/119,921

(22) PCT Filed: Oct. 13, 2023
(86) PCT No.: PCT/US2023/035069

§ 371 Date: Apr. 10, 2025

Related U.S. Application Data
(60) Provisional application No. 63/417,093, filed on Oct. 18, 2022.

(51) Int. Cl. C07D 473/00; A61K 31/444; A61K 31/52; C07D 471/04

(52) U.S. Cl. CPC C07D 473/00; A61K 31/444; A61K 31/52; C07D 471/04

(57) ABSTRACT

Disclosed herein are compounds of formulae (I), (II), and (III) and a pharmaceutically acceptable salts thereof. Also disclosed herein are uses of compounds of formulae (I), (II), and (III), as well as pharmaceutically acceptable salts thereof in the potential treatment or prevention of a disease or disorder which is modulated by the Kv3 potassium channels in a subject in need thereof. Also disclosed are compositions comprising one or more of the compounds. Further disclosed herein are uses of these compositions in the potential prevention or treatment of a disease or disorder which is modulated by the Kv3 potassium channels.

### BACKGROUND OF THE INVENTION

Voltage-dependent potassium (Kv) channels conduct potassium ions (K⁺) across cell membranes in response to changes in the membrane potential and can thereby regulate cellular excitability by modulating (increasing or decreasing) the electrical activity of the cell. Functional Kv channels exist as multimeric structures formed by the association of four alpha and four beta subunits. The alpha subunits comprise six transmembrane domains, a pore-forming loop and a voltage-sensor, and are arranged symmetrically around a central pore. The beta or auxiliary subunits interact with the alpha subunits and can modify the properties of the channel complex to include alterations in the channel's electrophysiological or biophysical properties, expression levels or expression patterns. Nine Kv channel alpha subunit families have been identified and are termed Kv1 through Kv9. As such, there is an enormous diversity in Kv channel function that arises as a consequence of the multiplicity of sub-families, the formation of both homomeric and heteromeric subunits within sub-families and the additional effects of association with beta subunits (Christie, 25 Clinical and Experimental Pharmacology and Physiology, 1995, 22, 944-951).

The Kv3 channel family consists of Kv3.1 (encoded by the KCNC1 gene), Kv3.2 (encoded by the KCNC2 gene), Kv3.3 (encoded by the KCNC3 gene) and Kv3.4 (encoded by the KCNC4 gene) (Rudy and McBain, 2001). Genes for each of these subtypes can generate multiple isoforms by alternative splicing, producing versions with different C-terminal domains. Kv3.1, Kv3.2 and Kv3.3 are prominently expressed in the central nervous system (CNS) whereas Kv3.4 expression pattern also includes peripheral nervous system (PNS) and skeletal muscle (Weiser et al. 1994). Although Kv3.1, Kv3.2 and Kv3.3 channels are broadly distributed in the brain (cerebellum, globus pallidus, subthalamic nucleus, thalamus, auditory brain stem, cortex and hippocampus), their expression is restricted to neuronal populations able to fire action potential (AP) of brief duration and to maintain high firing rates such as fast-spiking inhibitory interneurons (Rudy and McBain, 2001). Consequently, Kv3 channels display unique biophysical properties distinguishing them from other voltage-dependent potassium channels. Kv3 channels begin to open at relatively high membrane potentials (more positive than −20 mV) and exhibit rapid activation and deactivation kinetics (Kazmareck and Zhang, 2017). These characteristics ensure a fast repolarization and minimize the duration of after-hyperpolarization required for high frequency firing without affecting subsequent AP initiation and height.

Among Kv3 channels, Kv3.1 and Kv3.2 are particularly enriched in gabaergic interneurons including parvalbumin (PV) and somatostatin interneurons (SST) (Chow et al., 1999). Genetic ablation of Kv3.2 has been shown to broaden AP and to alter the ability to fire at high frequency in this neuronal population (Lau et al., 2000). Further, this genetic manipulation increased susceptibility to seizures. Similar phenotype was observed in mice lacking Kv3.1 and Kv3.3 confirming a crucial role of these channels in excitatory/inhibitory balance observed in epilepsy. This was confirmed at clinical level since several mutations within the KCNC1 (Kv3.1) gene have been shown to cause rare forms of epilepsy in human (Muona et al., 2015; Oliver et al., 2017). Consequently, positive modulators of Kv3 channel activators might restore excitatory/inhibitory imbalance, associated with epilepsy, through increasing the activity of inhibitory interneuron.

In addition to seizure susceptibility, excitatory/inhibitory imbalance has been postulated to participate in cognitive dysfunctions observed in a broad range of psychiatric disorders, including schizophrenia and autism spectrum disorder (Foss-Feig et al., 2017) as well as bipolar disorder, ADHD (Edden et al., 2012), anxiety-related disorders (Fuchs et al., 2017), and depression (Klempan et al., 2009). Post-mortem studies revealed alterations of certain gabaergic molecular markers in patients suffering from these pathologies (Straub et al., 2007; Lin and Sibille. 2013). Importantly, inhibition from parvalbumin and somatostatin interneurons projecting to the pyramidal excitatory neurons is important for the synchronized oscillatory activity of neural network, such as gamma oscillations (Bartos et al., 2007; Veit et al., 2017). This last type oscillation regulates diverse cognitive processes from sensory integration, attention, working memory and cognitive flexibility, domains that are particularly affected in psychiatric disorders (Herrmann and Demiralp; 2005). Therefore. Kv3 channel activators might rescue cognitive dysfunction and their associated alteration in gamma oscillations by increasing interneuron functions.

Both epileptiform activities and alterations of oscillations in the range of gamma have been observed at preclinical as well as clinical level in Alzheimer's disease (Palop and Mucke, 2016). While there is no current evidence of Kv3 channel alterations in Alzheimer's disease, Kv3 activators, through their actions on interneurons, could relieve both network alterations and cognitive abnormalities observed in this pathology and other neurodegenerative disorders.

Kv3.1 channels are particularly enriched in auditory brain stem. This particular neuronal population is required to fire AP at high rate (up to 600 Hz) and genetic ablation of Kv3.1 alters the ability of these neurons to follow high frequency stimulation (Macica et al., 2003). Kv3.1 levels in this structure has been shown to be altered in various conditions affecting auditory sensitivity, such as hearing loss (Von Hehn et al., 2004), fragile X (Strumbos et al., 2010) or tinnitus, suggesting that Kv3 activators have therapeutic potential in these disorders.

Kv3.4 channels, and to a lesser extent, Kv3.1, are expressed in the dorsal root ganglion (Tsantoulas and McMahon, 2014). Hypersensitivity to noxious stimuli in animal models of chronic pain have been associated with AP broadening (Chien et al., 2007). This phenomenon is partially due to alteration of Kv3.4 expression and function supporting the rationale to use Kv3 channels activator in the treatment of certain chronic pain conditions.

Kv3.1 and Kv3.2 are widely distributed within suprachiasmic nucleus, a structure responsible for controlling circadian rhythms. Mice lacking both Kv3.1 and Kv3.2 exhibit fragmented and altered circadian rhythm (Kudo et al., 2011). Consequently, Kv3.1 channel activators might be relevant for the treatment of sleep and circadian disorders, as well as sleep disruption as core symptom of psychiatric and neurodegenerative disorders.

KV3.1 channels are highly expressed in parvalbumin-positive interneurons located in the striatum (Munoz-Manchado et al., 2018). Although numerically rare compared to other neuronal populations of the striatum, they strongly influence striatal activity and consequently motoric function. Pharmacological inhibition of this population elicited dyskinetic movement, confirming their key role in motoric regulation and eventually in the pathophysiology of movement disorders (Gittis et al., 2011). Indeed, striatal parvalbumin interneuron alterations at both functional and density levels have been reported in numerous movement disorders including Huntington's disease (Lallani et al., 2019; Reiner et al., 2013), L-dopa-induced dyskinesia (Alberico et al., 2017), obsessive compulsive disorders (Burguiere et al., 2013), and Tourette syndrome (Kalanithi et al., 2005: Kataoka et al., 2010). Consequently, positive modulator of KV3 channels could exert attenuate abnormal movement observed in these pathologies through the modulation of striatal parvalbumin interneurons.

Although patients suffering from the above-mentioned disorders may have available treatment options, many of these options lack the desired efficacy and are accompanied by undesired side effects. Therefore, an unmet need exists for novel therapies for the treatment of these disorders.

Kv3 channels are also expressed by specific subsets of neurons in the spinal cord. Specifically, Kv3.1b (Deuchars et al., 2001; Brooke et al., 2002), Kv3.3 (Brooke et al., 2006), and Kv3.4 subunits (Brooke et al., 2004) have been identified in rodent spinal cord, although not always in association with circuits involved with sensory processing.

Recent animal model data suggest a down-regulation of Kv3.4 channel surface expression in DRG neurons following spinal cord injury associated with hypersensitivity to painful stimuli (Ritter et al., 2015). Similarly, it has been observed that there is a down-regulation of Kv3.4 expression in DRGs of rodents following spinal cord ligation (Chien et al., 2007). This latter study also showed that intrathecal administration to rats of an antisense oligonucleotide to suppress the expression of Kv3.4 led to hypersensitivity to mechanical stimuli. It has been shown that Kv3.4 channel inactivation could be influenced by protein kinase C-dependent phosphorylation of the channels, and that this physiological mechanism might allow DRG neurons to alter their firing characteristics in response to painful stimuli (Ritter et al., 2012). There is also evidence for changes in Kv3.4 expression in Alzheimer's disease (Hartmann et al., 2018). It has further been shown that Kv3.1 and Kv3.4 are involved in cancer cell migration and invasion (Song et al., 2018).

These studies suggest a causal relationship between the emergence of mechanical allodynia and reduced Kv3.4 channel expression or function. No evaluation of Kv3.1, Kv3.2, or Kv3.3 expression in SC or DRG neurons was conducted in any of these studies, and expression of these three subtypes has not been explicitly demonstrated on DRG neurons (although as mentioned above, they are abundant within specific regions of the spinal cord).

There remains a need for the identification of alternative modulators for the prophylaxis or treatment of pain which may display one or more of the following desirable properties: improved efficacy; improved potency; more convenient administration regimes; reduced side-effect burden; reduced tolerance or tachyphylaxis; and reduced abuse liability and/or risk of dependence.

Modulation of one or more of Kv3.1, Kv3.2 and Kv3.3 channels is linked to the processing of pain and pain control. Therefore, modulation of Kv3.1, Kv3.2 and/or Kv3.3 represents a new approach for the prophylaxis or treatment of pain.

### SUMMARY OF THE INVENTION

Disclosed herein are novel compounds of formulae (I), (II), and (III) and their pharmaceutically acceptable salts. Also disclosed herein are uses of these compounds in the potential treatment or prevention of a disorder which is modulated by the Kv3 potassium channels. Also disclosed herein are compositions comprising one or more of the compounds. Further disclosed herein are uses of these compositions in the potential prevention or treatment of a disorder which is modulated by the Kv3 potassium channels.

### DETAILED DESCRIPTION OF THE INVENTION

Disclosed herein is a compound of formula (I), (II), or (III) or a pharmaceutically acceptable salt thereof:

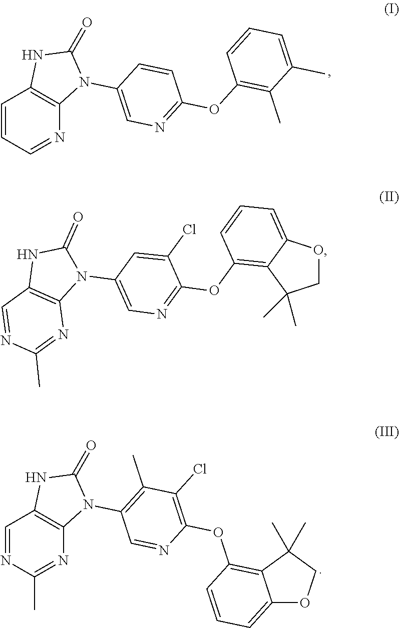 [SMILES: Cc1cccc(Oc2ccc(-n3c(=O)[nH]c4cccnc43)cn2)c1C ; Cc1ncc2[nH]c(=O)n(-c3cnc(Oc4cccc5c4C(C)(C)CO5)c(Cl)c3)c2n1 ; Cc1ncc2[nH]c(=O)n(-c3cnc(Oc4cccc5c4C(C)(C)CO5)c(Cl)c3C)c2n1]

Also disclosed herein is a pharmaceutical composition comprising a compound of formula (I), (II), or (III) or a pharmaceutically acceptable salt thereof, and a pharmaceutically acceptable carrier.

Also disclosed herein is a method for treating a disease or disorder of a subject which is modulated by Kv3 potassium channels comprising administering a compound of formula (I), (II), or (III) or a pharmaceutically acceptable salt thereof.

In one embodiment, the disease or disorder modulated by Kv3 potassium channels is a neurological or psychiatric disorder selected from epilepsy, schizophrenia, schizophreniform disorder, schizoaffective disorder, cognitive impairment associated with schizophrenia (CIAS), autism spectrum disorder, bipolar disorder, attention deficit hyperactivity disorder (ADHD), anxiety-related disorder, depression, cognitive dysfunction. Alzheimer's disease, fragile X syndrome, chronic pain, hearing loss, sleep disorder, sleep disruption, Huntington's disease, Parkinson's disease, L-dopa-induced dyskinesia, obsessive-compulsive disorder, and Tourette's syndrome.

In one embodiment, the disease or disorder modulated by Kv3 potassium channels is schizophrenia of the paranoid, disorganized, catatonic, undifferentiated, or residual type.

In one embodiment, the disease or disorder modulated by Kv3 potassium channels is a schizoaffective disorder of the delusional or depressive type.

Further disclosed herein is the use of a compound of formula (I), (II), or (III) or a pharmaceutically acceptable salt thereof, for the manufacture of a medication for the treatment of a disease or disorder which is modulated by Kv3 potassium channels.

As used herein, “alkyl” refers to both branched- and straight-chain saturated aliphatic hydrocarbon groups of 1 to 18 carbon atoms, or more specifically, 1 to 12 carbon atoms. Examples of such groups include, but are not limited to, methyl (Me), ethyl (Et), n-propyl (Pr), n-butyl (Bu), n-pentyl, n-hexyl, and the isomers thereof such as isopropyl (i-Pr), isobutyl (i-Bu), sec-butyl (s-Bu), tert-butyl (t-Bu), isopentyl, and isohexyl. Alkyl groups may be optionally substituted with one or more substituents as defined herein. “C₁₋₆alkyl” refers to an alkyl group as defined herein having 1 to 6 carbon atoms.

“Halo” or “halogen” refers to fluoro, chloro, bromo or iodo, unless otherwise noted. In one embodiment, halogen is selected from fluoro, chloro and bromo. In one embodiment, halogen is selected from chloro and bromo. In one embodiment, halogen is bromo. In one embodiment, halogen is chloro.

“Optionally substituted” refers to “unsubstituted or substituted,” and therefore, the generic structural formulas described herein encompass compounds containing the specified optional substituent(s) as well as compounds that do not contain the optional substituent(s). Each substituent is independently defined each time it occurs within the generic structural formula definitions.

### Polymorphism

A compound disclosed herein, including a salt thereof, may exist in crystalline form, non-crystalline form, or a mixture thereof. A compound or a salt thereof may also exhibit polymorphism. i.e., the capacity of occurring in different crystalline forms. These different crystalline forms are typically known as “polymorphs”. Polymorphs have the same chemical composition but differ in packing, geometrical arrangement, and other descriptive properties of crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, all of which may be used for identification. One of ordinary skill in the art will appreciate that different polymorphs may be produced, for example, by changing or adjusting the conditions used in crystallizing/recrystallizing a compound disclosed herein.

### Optical Isomers-Diastereomers-Geometric Isomers-Tautomers

Included herein are various isomers of the compounds disclosed herein. The term “isomers” refers to compounds that have the same composition and molecular weight but differ in physical and/or chemical properties. The structural difference may be in constitution (geometric isomers) or in the ability to rotate the plane of polarized light (stereoisomers).

With regard to stereoisomers, a compound disclosed herein may have one or more asymmetric carbon atom and may occur as mixtures (such as a racemic mixture) or as individual enantiomers or diastereomers. All such isomeric forms are included herein, including mixtures thereof. If a compound disclosed herein contains a double bond, the substituent may be in the E or Z configuration. If a compound disclosed herein contains a disubstituted cycloalkyl, the cycloalkyl substituent may have a cis- or trans-configuration. All tautomeric forms are also intended to be included.

Any asymmetric atom (e.g., carbon) of a compound disclosed herein, can be present in racemic mixture or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis- (Z)- or trans- (E)-form.

A compound disclosed herein, can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of the final compounds of the examples or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds can also be resolved by chiral chromatography, e.g., high pressure liquid chromatography (HPLC) using a chiral adsorbent.

Some of the compounds described herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are included within the scope of the present invention.

### Isotopic Variations

Compounds disclosed herein, include unlabeled forms, as well as isotopically labeled forms. Isotopically labeled compounds have structures depicted by the formulas given herein except that one or more atoms are replaced by an atom having a selected atomic mass or mass number. Examples of isotopes that can be incorporated into compounds disclosed herein include isotopes of hydrogen, carbon, nitrogen, oxygen, phosphorous, sulfur, fluorine, iodine and chlorine, such as ²H (i.e., Deuterium or “D”), ³H, ¹¹C, ¹³C, ¹⁴C, ¹³N, ¹⁵N, ¹⁵O, ¹⁷O, ¹⁸O, ³²P, ³⁵S, ¹⁸F, ¹²³I, ¹²⁵I and ³⁶Cl. The invention includes various isotopically labeled compounds as defined herein, for example those into which radioactive isotopes, such as ³H and ¹⁴C, or those into which non-radioactive isotopes, such as ²H and ¹³C are present. Such isotopically labeled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, substitution with positron emitting isotopes, such as ¹¹C, ¹⁸F, ¹⁵O and ¹³N, may be particularly desirable for PET or SPECT studies.

Isotopically-labeled compounds disclosed herein, can generally be prepared by conventional techniques known to those skilled in the art. Furthermore, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index.

### Pharmaceutically Acceptable Salts

The term “pharmaceutically acceptable salt” refers to a salt prepared from a pharmaceutically acceptable non-toxic base or acid, including inorganic or organic base and inorganic or organic acid. Salts derived from inorganic bases include aluminum, ammonium, calcium, copper, ferric, ferrous, lithium, magnesium, manganic salts, manganous, potassium, sodium, zinc, and the like. Particular embodiments include ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When a compound disclosed herein is basic, a salt may be prepared from a pharmaceutically acceptable non-toxic acid, including an inorganic and organic acid. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, trifluoroacetic acid (TFA) and the like. Particular embodiments include the citric, hydrobromic, hydrochloric, maleic, phosphoric, sulfuric, fumaric, tartaric and trifluoroacetic acids.

### Methods of Use

The present disclosure provides compounds of formula (I), (II), or (III) or a pharmaceutically acceptable salt thereof for use in therapy.

The compounds of formula (I), (II), or (III) or their pharmaceutically acceptable salts may be used for treating a disease or disorder where a modulator of the Kv3.1, Kv3.2, or Kv3.1 and Kv3.2 channels is required. As used herein, a modulator of Kv3.1, Kv3.2, or Kv3.1 and Kv3.2 is a compound which alters the properties of these channels, either positively or negatively. The altered property of the channel may be the scale of response observed or the temporal behaviour of the channel.

Compounds of the invention may be tested in the biological assays described below in the Experimental section to determine their modulatory properties.

In certain disorders it may be of benefit to utilise a modulator of Kv3.1 or Kv3.2 which demonstrates a particular selectivity profile between the two channels. For example a compound may be selective for modulation of Kv3.1 channels over modulation of Kv3.2 channels demonstrating, for example, at least a 2 fold, 5 fold or 10 fold activity for Kv3.1 channels than for Kv3.2 channels. Alternatively, a compound may be selective for modulation of Kv3.2 channels over modulation of Kv3.1 channels demonstrating, for example, at least a 2 fold, 5 fold or 10 fold activity for Kv3.2 channels than for Kv3.1 channels. In other cases a compound may demonstrate comparable activity between modulation of Kv3.1 and Kv3.2 channels, for example the activity for each channel is less than 2 fold that for the other channel, such as less than 1.5 fold or less than 1.2 fold. The activity of a compound is suitably quantified by its potency as indicated by an EC₅₀ value.

Diseases or conditions that may be mediated by modulation of Kv3.1 and/or Kv3.2 channels may be selected from the list below. The numbers in brackets after the listed diseases below refer to the classification code in Diagnostic and Statistical Manual of Mental Disorders, 5th Edition, published by the American Psychiatric Association (DSM-V) and/or the International Classification of Diseases, 10th Edition (ICD-10).

The compounds of formula (I), (II), or (III) or their pharmaceutically acceptable salts may be of use for the treatment or prophylaxis of depression and mood disorders including Major Depressive Episode, Manic Episode, Mixed Episode and Hypomanic Episode; Depressive Disorders including Major Depressive Disorder, Dysthymic Disorder (F34.1), Depressive Disorder Not Otherwise Specified (F32.8); Bipolar Disorders including Bipolar I Disorder, Bipolar II Disorder (Recurrent Major Depressive Episodes with Hypomanic Episodes) (F31.81), Cyclothymic Disorder (F34.0) and Bipolar Disorder Not Otherwise Specified (F31.9); Other Mood Disorders including Mood Disorder Due to a General Medical Condition which includes the subtypes With Depressive Features, With Major Depressive-like Episode, With Manic Features (F06.33) and With Mixed Features (F06.34), Substance-Induced Mood Disorder (including the subtypes With Depressive Features, With Manic Features and With Mixed Features) and Mood Disorder Not Otherwise Specified (F39.0); Seasonal affective disorder.

The compounds of formula (I), (II), or (III) or their pharmaceutically acceptable salts may be of use for the treatment or prophylaxis of schizophrenia including the subtypes Paranoid Type (F20.0), Disorganised Type (F20.1), Catatonic Type (F20.2), Undifferentiated Type (F20.3) and Residual Type (F20.5); Schizophreniform Disorder (F20.81); Schizoaffective Disorder (F25.9) including the subtypes Bipolar Type (F25.0) and Depressive Type (F25.1); Delusional Disorder (F22.0) including the subtypes Erotomanic Type, Grandiose Type, Jealous Type, Persecutory Type, Somatic Type, Mixed Type and Unspecified Type; Brief Psychotic Disorder (F23.0); Shared Psychotic Disorder (F24.0): Psychotic Disorder Due to a General Medical Condition including the subtypes With Delusions (F06.2) and With Hallucinations (F06.0); Substance-Induced Psychotic Disorder including the subtypes With Delusions (F06.2) and With Hallucinations (F06.0); and Psychotic Disorder Not Otherwise Specified (F29.0).

The compounds of formula (I), (II), or (III) or their pharmaceutically acceptable salts may be of use for the treatment or prophylaxis of anxiety disorders including Panic Attack; Panic Disorder including Panic Disorder without Agoraphobia (F41.0) and Panic Disorder with Agoraphobia (F40.01); Agoraphobia; Agoraphobia Without History of Panic Disorder (F40.00), Specific Phobia (F40.20, formerly Simple Phobia) including the subtypes Animal Type (F40.21), Natural Environment Type (F40.22), Blood-Injection-Injury Type (F40.23), Situational Type (F40.24) and Other Type (F40.29), Social Phobia (Social Anxiety Disorder, F40.10), Obsessive-Compulsive Disorder (F42.0), Posttraumatic Stress Disorder (F43.10), Acute Stress Disorder (F43.0), Generalized Anxiety Disorder (F41.1), Anxiety Disorder Due to a General Medical Condition (F06.4), Substance-Induced Anxiety Disorder, Separation Anxiety Disorder (F93.0), Adjustment Disorders with Anxiety (F43.22) and Anxiety Disorder Not Otherwise Specified (F41.9).

The compounds of formula (I), (II), or (III) or their pharmaceutically acceptable salts may be of use for the enhancement of cognition including the treatment of cognition impairment in other diseases such as schizophrenia, bipolar disorder, depression, other psychiatric disorders and psychotic conditions associated with cognitive impairment, e.g., Alzheimer's disease. Alternatively, the compounds of formula (I), (II), or (III) or their pharmaceutically acceptable salts and/or solvates may be of use for the prophylaxis of cognition impairment, such as may be associated with diseases such as schizophrenia, bipolar disorder, depression, other psychiatric disorders and psychotic conditions associated with cognitive impairment, e.g., Alzheimer's disease.

The compounds of formula (I), (II), or (III) or their pharmaceutically acceptable salts may be of use for the treatment or prophylaxis of sleep disorders including primary sleep disorders such as Dyssomnias such as Primary Insomnia (G47.00), Primary Hypersomnia (F51.11), Narcolepsy (G47.419), Breathing-Related Sleep Disorders (G47.9), Circadian Rhythm Sleep Disorder (F51.8) and Dyssomnia Not Otherwise Specified (F51.8); primary sleep disorders such as Parasomnias such as Nightmare Disorder (F51.5), Sleep Terror Disorder (F51.4), Sleepwalking Disorder (F51.3) and Parasomnia Not Otherwise Specified (F51.8); Sleep Disorders Related to Another Mental Disorder such as Insomnia Related to Another Mental Disorder (F51.19) and Hypersomnia Related to Another Mental Disorder (F51.19); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type: sleep apnea and jet-lag syndrome.

The compounds of formula (I), (II), or (III) or their pharmaceutically acceptable salts may be of use for the treatment or prophylaxis of Autism Spectrum Disorders including Autistic Disorder (F84.0), Asperger's Disorder (F84.5), Rett's Disorder (F84.8), Childhood Disintegrative Disorder (F84.3) and Pervasive Disorder Not Otherwise Specified (F84.8, including Atypical Autism).

The compounds of formula (I), (II), or (III) or their pharmaceutically acceptable salts may be of use for the treatment or prophylaxis of Attention-Deficit/Hyperactivity Disorder including the subtypes Attention-Deficit/Hyperactivity Disorder Combined Type (F90.2), Attention-Deficit/Hyperactivity Disorder Predominantly Inattentive Type (F90.0), Attention-Deficit/Hyperactivity Disorder Hyperactive-Impulse Type (F90.1) and Attention-Deficit/Hyperactivity Disorder Not Otherwise Specified (F90.9); Hyperkinetic Disorder; Disruptive Behaviour Disorders such as Conduct Disorder including the subtypes childhood-onset type (F91.1). Adolescent-Onset Type (F91.2) and Unspecified Onset (F91.9), Oppositional Defiant Disorder (F91.3) and Disruptive Behaviour Disorder Not Otherwise Specified; and Tic Disorders such as Tourette's Disorder (F95.2).

The compounds of formula (I), (II), or (III) or their pharmaceutically acceptable salts may be of use for the treatment or prophylaxis of Personality Disorders including the subtypes Paranoid Personality Disorder (F60.0), Schizoid Personality Disorder (F60.1), Schizotypal Personality Disorder (F21.0), Antisocial Personality Disorder (F60.2), Borderline Personality Disorder (F60.3), Histrionic Personality Disorder (F60.4), Narcissistic Personality Disorder (F60.81), Avoidant Personality Disorder (F60.6), Dependent Personality Disorder (F60.7), Obsessive-Compulsive Personality Disorder (F60.5) and Personality Disorder Not Otherwise Specified (F60.9).

The compounds of formula (I), (II), or (III) or their pharmaceutically acceptable salts may be of use for the treatment or prophylaxis of Impulse control disorder including: Intermittent Explosive Disorder (F63.81), Kleptomania (F63.2), Pathological Gambling (F63.0), Pyromania (F63.1), Trichotillomania (F63.3), Impulse-Control Disorders Not Otherwise Specified (F63.9), Binge-Eating Disorder (F50.8). Compulsive Buying. Compulsive Sexual Behaviour and Compulsive Hoarding.

The compounds of formula (I), (II), or (III) or their pharmaceutically acceptable salts may be of use for the treatment or prophylaxis of hearing disorders including auditory neuropathy, auditory processing disorder, hearing loss, which includes sudden hearing loss, noise induced hearing loss, substance-induced hearing loss, and hearing loss in adults over 60 (presbycusis), and tinnitus.

The compounds of formula (I), (II), or (III) or their pharmaceutically acceptable salts may be of use for the treatment or prophylaxis of Ménière's disease, disorders of balance, and disorders of the inner ear.

The compounds of formula (I), (II), or (III) or their pharmaceutically acceptable salts may be of use for the treatment or prophylaxis of hyperacusis and disturbances of loudness perception, including Fragile-X syndrome and autism.

The compounds of formula (I), (II), or (III) or their pharmaceutically acceptable salts may be of use for the treatment or prophylaxis of epilepsy, (including, but not limited to, localization-related epilepsies, generalized epilepsies, epilepsies with both generalized and local seizures, and the like), seizures associated with Lennox-Gastaut syndrome, seizures as a complication of a disease or condition (such as seizures associated with encephalopathy, phenylketonuria, juvenile Gaucher's disease, Lundborg's progressive myoclonic epilepsy, stroke, head trauma, stress, hormonal changes, drug use or withdrawal, alcohol use or withdrawal, sleep deprivation, fever, infection, and the like), essential tremor, restless limb syndrome, partial and generalised seizures (including tonic, clonic, tonic-clonic, atonic, myoclonic, absence seizures), secondarily generalized seizures, temporal lobe epilepsy, absence epilepsies (including childhood, juvenile, myoclonic, photo- and pattern-induced), severe epileptic encephalopathies (including hypoxia-related and Rasmussen's syndrome), febrile convulsions, epilepsy partialis continua, progressive myoclonus epilepsies (including Unverricht-Lundborg disease and Lafora's disease), post-traumatic seizures/epilepsy including those related to head injury, simple reflex epilepsies (including photosensitive, somatosensory and proprioceptive, audiogenic and vestibular), metabolic disorders commonly associated with epilepsy such as pyridoxine-dependent epilepsy, Menkes' kinky hair disease. Krabbe's disease, epilepsy due to alcohol and drug abuse (e.g. cocaine), cortical malformations associated with epilepsy (e.g. double cortex syndrome or subcortical band heterotopia), chromosomal anomalies associated with seizures or epilepsy such as Partial monosomy (15Q)/Angelman syndrome.

In one embodiment, there is provided a compound of formula (I), (II), or (III) or a pharmaceutically acceptable salt thereof for the treatment or prophylaxis of depression and mood disorders, hearing disorders, schizophrenia, substance abuse disorders, sleep disorders or epilepsy.

In one embodiment, there is provided a compound of formula (I), (II), or (III) or a pharmaceutically acceptable salt thereof for the treatment or prophylaxis of bipolar disorder or mania.

In one embodiment, there is provided a compound of formula (I), (II), or (III) or a pharmaceutically acceptable salt and/or solvate thereof for the treatment or prophylaxis of ataxia, such as spinocerebellar ataxia.

In one embodiment, disclosed herein is a method for treating a disease or disorder which is modulated by Kv3 potassium channels. In one embodiment, the method comprises administering an effective amount of a compound of formula (I), (II), or (III) or a pharmaceutically acceptable salt thereof, to a subject suffering from a disease or disorder which is modulated by Kv3 potassium channels. In one embodiment, the disease or disorder which is modulated by Kv3 potassium channels is selected from epilepsy, schizophrenia, schizoaffective disorder, cognitive impairment associated with schizophrenia, bipolar disorder, attention deficit hyperactivity disorder (ADHD), anxiety, depression, cognitive dysfunction, Alzheimer's disease, hearing loss, tinnitus, fragile X syndrome, pain, sleep disorder, circadian disorder, and sleep disruption and movement disorder, such as Huntington's disease, Parkinson's disease, L-dopa-induced dyskinesia, obsessive compulsive disorders, and Tourette syndrome.

In one embodiment, a compound of formula (I), (II), or (III), or a pharmaceutically acceptable salt thereof, is for use as a medicament. In one embodiment, the medicament is for use in treating or alleviating epilepsy, schizophrenia, schizoaffective disorder, cognitive impairment associated with schizophrenia, bipolar disorder, ADHD, anxiety, depression, cognitive dysfunction, Alzheimer's disease, hearing loss, tinnitus, fragile X syndrome, pain, sleep disorder, circadian disorder, or sleep disruption and movement disorders, such as Huntington's disease, Parkinson's disease, L-dopa-induced dyskinesia, obsessive compulsive disorders, and Tourette syndrome.

In another embodiment, a compound of formula (I), (II), or (III) or a pharmaceutically acceptable salt thereof, is for the manufacture of a medicament for the treatment of epilepsy, schizophrenia, schizoaffective disorder, cognitive impairment associated with schizophrenia, bipolar disorder, ADFID, anxiety, depression, cognitive dysfunction, Alzheimer's disease, hearing loss, tinnitus, fragile X syndrome, pain, sleep disorder, circadian disorder, sleep disruption and movement disorders, such as Huntington's disease, Parkinson's disease, L-dopa-induced dyskinesia, obsessive compulsive disorders, and Tourette syndrome.

### Administration Routes

The compounds of formula (I), (II), or (III) or their pharmaceutically acceptable salts may be administered by any convenient method, e.g., by oral, parenteral, buccal, sublingual, nasal, rectal or transdermal administration, and the pharmaceutical compositions adapted accordingly.

The compounds of formula (I), (II), or (III) or their pharmaceutically acceptable salts which are active when given orally can be formulated as liquids or solids, e.g., as syrups, suspensions, emulsions, tablets, capsules or lozenges.

A liquid formulation will generally consist of a suspension or solution of the active ingredient in a suitable liquid carrier(s) e.g., an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g., pellets containing the active ingredient can be prepared using standard carriers and then filled into a hard gelatin capsule; alternatively a dispersion or suspension can be prepared using any suitable pharmaceutical carrier(s), e.g., aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g., polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas, e.g., air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

In one embodiment the composition is in unit dose form such as a tablet, capsule or ampoule.

### Doses

The amount of a compound of formula (I), (II), or (III) or a pharmaceutically acceptable salt thereof, administered to a subject is an amount sufficient to modulate Kv3 potassium channels in the subject. In one embodiment, the amount of a compound of formula (I), (II), or (III) or a pharmaceutically acceptable salt thereof, can be an “effective amount”, wherein the subject compound is administered in an amount that will elicit a biological or medical response of a tissue, system, animal or human that is being sought by a researcher, veterinarian, medical doctor or other clinician. An effective amount does not necessarily include considerations of toxicity and safety related to the administration of a compound. It is recognized that one skilled in the art may affect physiological disorders associated with Kv3 potassium channels by treating a subject presently afflicted with the disorders, or by prophylactically treating a subject likely to be afflicted with the disorders, with an effective amount of a compound of formula (I), (II), or (III) or a pharmaceutically acceptable salt thereof.

An effective amount of a compound will vary with the particular compound chosen (e.g. considering the potency, efficacy, and/or half-life of the compound); the route of administration chosen; the condition being treated; the severity of the condition being treated; the age, size, weight, and physical condition of the subject being treated; the medical history of the subject being treated; the duration of the treatment; the nature of a concurrent therapy; the desired therapeutic effect; and like factors and can be routinely determined by the skilled artisan.

In one embodiment, a compound of formula (I), (II), or (III) or a pharmaceutically acceptable salt thereof, is administered in an amount from about 0.001 mg/kg body weight to about 100 mg/kg body weight per day. In one embodiment, daily dosages may be in the range of 0.01 mg/kg body weight to about 50 mg/kg body weight per day; or more specifically, in the range of 0.1 mg/kg body weight to about 25 mg/kg body weight per day. The exact dosages will depend upon the frequency and mode of administration, the gender, the age, the weight, and the general condition of the subject to be treated, the nature and the severity of the condition to be treated, any concomitant diseases to be treated, the desired effect of the treatment and other factors known to those skilled in the art.

A typical oral dosage for adults will be in the range of 0.1-1000 mg/day of a compound of formula (I), (II), or (III) or a pharmaceutically acceptable salt thereof; or more specifically, 1-500 mg/day; or more specifically, 1-100 mg/day; or even more specifically. 1-50 mg/day. Conveniently, a compound of formula (I), (II), or (III) or a pharmaceutically acceptable salt thereof, is administered in a unit dosage form containing said compound in an amount of about 0.1 to 500 mg, such as 10 mg, 50 mg 100 mg, 150 mg, 200 mg or 250 mg.

A subject administered with a compound of formula (I), (II), or (III), or a pharmaceutically acceptable salt thereof, is generally a mammal, such as a human being, male or female. A subject also refers to cows, sheep, goats, horses, dogs, cats, rabbits, rats, mice, fish, and birds. In one embodiment, the subject is a human.

As used herein, the terms “treatment” and “treating” refer to all processes wherein there is a slowing, interrupting, arresting, controlling, or stopping of the progression of a disease or disorder which is modulated by Kv3 potassium channels. The terms do not necessarily indicate a total elimination of all disease or disorder symptoms.

The terms “administration of” and or “administering a” compound should be understood to include providing a compound described herein, or a pharmaceutically acceptable salt, solvate or hydrate thereof, and compositions of the foregoing to a subject.

### Compositions

The term “composition” as used herein is intended to encompass a dosage form comprising a specified compound in a specified amount, as well as any dosage form which results, directly or indirectly, from a combination of a specified compound in a specified amount. Such term is intended to encompass a dosage form comprising a compound disclosed herein, or a pharmaceutically acceptable salt thereof, and one or more pharmaceutically acceptable carriers or excipients. Accordingly, the compositions of the present invention encompass any composition made by admixing a compound of the present invention and one or more pharmaceutically acceptable carrier or excipients. By “pharmaceutically acceptable” it is meant the carriers or excipients are compatible with the compound disclosed herein and with other ingredients of the composition.

In one embodiment, disclosed herein is a composition comprising a compound of formula (I), (II), or (III) or a pharmaceutically acceptable salt thereof, and a pharmaceutically acceptable carrier or excipient. The composition may be prepared and packaged in bulk form wherein an effective amount of a compound of the invention can be extracted and then given to a subject, such as with powders or syrups. Alternatively, the composition may be prepared and packaged in unit dosage form wherein each physically discrete unit contains an effective amount of a compound disclosed herein. When prepared in unit dosage form, the composition of the invention typically contains from about 0.1 mg to 2 grams, or more specifically, 0.1 mg to 500 mg, or even more specifically, 0.2 mg to 100 mg, of a compound of formula (I), (II), or (III) or a pharmaceutically acceptable salt thereof.

A compound disclosed herein and a pharmaceutically acceptable carrier or excipient(s) will typically be formulated into a dosage form adapted for administration to a subject by a desired route of administration. For example, dosage forms include those adapted for (1) oral administration, such as tablets, capsules, caplets, pills, troches, powders, syrups, elixirs, suspensions, solutions, emulsions, sachets, and cachets; and (2) parenteral administration, such as sterile solutions, suspensions, and powders for reconstitution. Suitable pharmaceutically acceptable carriers or excipients will vary depending upon the particular dosage form chosen. In addition, suitable pharmaceutically acceptable carriers or excipients may be chosen for a particular function that they may serve in the composition. For example, certain pharmaceutically acceptable carriers or excipients may be chosen for their ability to facilitate the production of uniform dosage forms. Certain pharmaceutically acceptable carriers or excipients may be chosen for their ability to facilitate the production of stable dosage forms. Certain pharmaceutically acceptable carriers or excipients may be chosen for their ability to facilitate the carrying or transporting of a compound disclosed herein, once administered to the subject, from one organ or portion of the body to another organ or another portion of the body. Certain pharmaceutically acceptable carriers or excipients may be chosen for their ability to enhance patient compliance.

Suitable pharmaceutically acceptable excipients include the following types of excipients: diluents, lubricants, binders, disintegrants, fillers, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents.

A skilled artisan possesses the knowledge and skill in the art to select suitable pharmaceutically acceptable carriers and excipients in appropriate amounts for the use in the invention. In addition, there are a number of resources available to the skilled artisan, which describe pharmaceutically acceptable carriers and excipients and may be useful in selecting suitable pharmaceutically acceptable carriers and excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The compositions of the invention are prepared using techniques and methods known to those skilled in the art. Some methods commonly used in the art are described in Remington's Pharmaceutical Sciences (Mack Publishing Company).

In one embodiment, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives, (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch) gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmellose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. The composition can also be prepared to prolong or sustain the release as, for example, by coating or embedding particulate material in polymers, wax, or the like.

The compounds disclosed herein may also be coupled with soluble polymers as targetable drug carriers. Such polymers can include polyvinylpyrrolidone, pyrancopolymer, polyhydroxypropylmethacrylamidephenol, polyhydroxyethylaspartamidephenol, or polyethyleneoxidepolylysine substituted with palmitoyl residues. Furthermore, the compounds of the invention may be coupled to a class of biodegradable polymers useful in achieving controlled release of a drug, for example polylactic acid, polyepsilon caprolactone, polyhydroxy butyric acid, polyorthoesters, polyacetals, polydihydropyrans, polycyanoacrylates and cross-linked or amphipathic block copolymers of hydrogels.

In one embodiment, the invention is directed to a liquid oral dosage form. Oral liquids such as solution, syrups and elixirs can be prepared in dosage unit form so that a given quantity contains a predetermined amount of a compound disclosed herein. Syrups can be prepared by dissolving the compound of the invention in a suitably flavored aqueous solution; while elixirs are prepared through the use of a non-toxic alcoholic vehicle. Suspensions can be formulated by dispersing a compound disclosed herein in a non-toxic vehicle. Solubilizers and emulsifiers such as ethoxylated isostearyl alcohols and polyoxy ethylene sorbitol ethers, preservatives, flavor additives such as peppermint oil or other natural sweeteners or saccharin or other artificial sweeteners and the like can also be added.

In one embodiment, the invention is directed to compositions for parenteral administration. Compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

### EXPERIMENTAL

The following synthetic schemes and examples are intended to be illustrative only and not limiting in any way. Abbreviations used are those conventional in the art or as defined below.
aq. Aqueous
° C. degree celsius
DMF N,N-dimethylformamide
DMSO dimethylsulfoxide
EA ethyl acetate
Et ethyl
EtOAc ethyl acetate
EtOH ethanol
g gram
h hour(s)
Hex hexanes
HPLC high pressure liquid chromatography
kg kilogram
L liter
LC liquid chromatography
LCMS liquid chromatography and mass spectrometry
LRMS high resolution mass spectrometry
M molar
Me methyl
MeCN acetonitrile
mg milligram
mmol millimole
MS mass spectrometry
MTBE methyl tert-butyl ether
min minutes
mL milliliter(s)
m/z mass to charge ratio
nm nanometer
nM nanomolar
N normal
PDL Poly-D-lysine
PE petroleum ether
PG protecting group
RT or rt room temperature
sat. saturated
TFA trifluoroacetic acid
THF tetrahydrofuran
uL microliter(s)
tBuXPhos Pd G3 [(2-Di-tert-butylphosphino-2′,4′,6′-triisopropyl-1,1′-biphenyl)-2-(2′-amino-1,1′-biphenyl)]palladium(II) methanesulfonate

### General Synthetic Schemes

The following syntheses are representative exemplary methods by which the compounds disclosed herein can be synthesized.

In the schemes described below, it is well understood that protecting groups for sensitive or reactive groups are employed where necessary in accordance with general principles of chemistry. Protecting groups are manipulated according to standard methods of organic synthesis (P. G. M. Wuts, “Greene's Protective Groups in Organic Synthesis”, Wiley, New York 2014). These groups are removed at a convenient stage of the compound synthesis using methods that are readily apparent to those skilled in the art.

Starting materials are either commercially available or made by known procedures in the literature or as illustrated. In some cases the order of carrying out the reaction schemes may be varied to facilitate the reaction or to avoid unwanted reaction products. The following examples are provided for the purpose of illustration only and are not to be construed as limitations on the disclosed invention.

All syntheses may be performed using reagents and reaction conditions suitable for similar reactions known in the field. Compounds disclosed herein may be produced by employing analogous syntheses.

Reagents were purchased from commercial sources and were used as received. ¹H NMR spectra were obtained on a Bruker AVANCE 300 spectrometer at 300 MHz and a Bruker AVANCE 400 spectrometer at 400 MHz with tetramethylsilane used as an internal reference. The mass spectra were obtained on a Finnigan LCQ-DUO spectrometer and Shimadzu LCMS-2020 spectrometer using electrospray ionization. HPLC analyses were performed on an Agilent 1100 Series instrument and on a Shimadzu SIL-20A instrument.

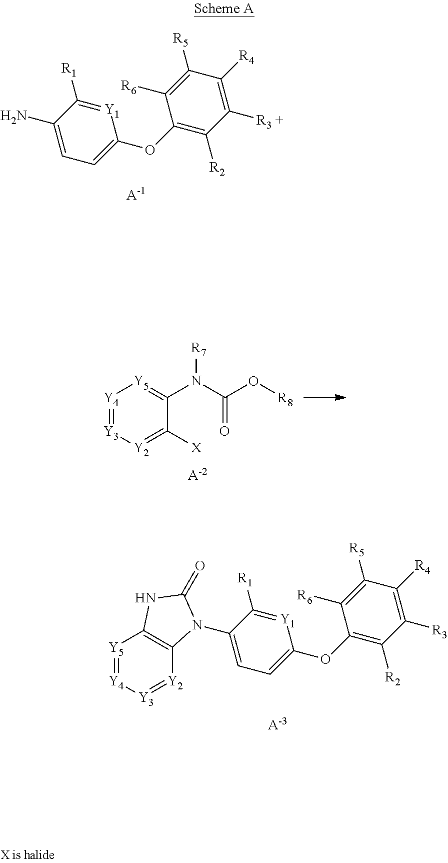

As illustrated in Scheme A, compounds disclosed herein can generally be prepared by palladium-catalyzed N-arylation between an appropriately functionalized aminoheterocycle A-1 and aromatic or heteroaromatic halide A-2, with subsequent one-pot cyclization on the carbamate carbonyl to afford compounds of formula A-3.

### Intermediates

### Intermediate 1. 5-chloro-6-((2,3-dihydrobenzofuran-4-yl)oxy)-4-methylpyridin-3-amine

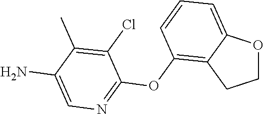 [SMILES: Cc1c(N)cnc(Oc2cccc3c2CCO3)c1Cl]

### Step A: 3-chloro-2-((2,3-dihydrobenzofuran-4-yl)oxy)-4-methyl-5-nitropyridine

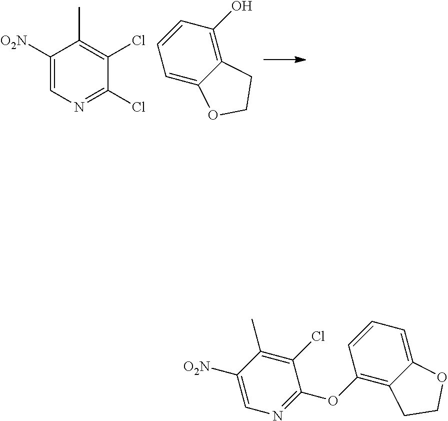

To a mixture of 2,3-dihydrobenzofuran-4-ol (145 mg, 1.065 mmol) in DMF (3994 μl)/Water (1331 μl) was added K₂CO₃ (294 mg, 2.130 mmol) followed by 2,3-dichloro-4-methyl-5-nitropyridine (231 mg, 1.118 mmol). The resulting mixture was heated to 55° C. overnight. The reaction mixture was concentrated under vacuum and purified by silica gel chromatography (0-100% EA/Hex) to give the title compound.

### Step B: 5-chloro-6-((2,3-dihydrobenzofuran-4-yl)oxy)-4-methylpyridin-3-amine

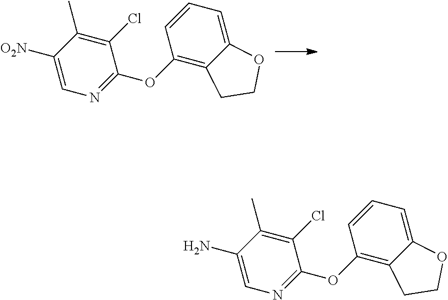 [SMILES: Cc1c([N+](=O)[O-])cnc(Oc2cccc3c2CCO3)c1Cl ; Cc1c(N)cnc(Oc2cccc3c2CCO3)c1Cl]

To a flask containing 3-chloro-2-((2,3-dihydrobenzofuran-4-yl)oxy)-4-methyl-5-nitropyridine (136 mg. 0.443 mmol) was added iron (248 mg, 4.43 mmol) and ammonium chloride (119 mg, 2.217 mmol) followed by THF (5853 μl), Ethanol (1153 μl), water, and acetic acid (25.4 μl, 0.443 mmol). The resulting mixture was stirred overnight. The mixture was filtered, poured into sat aq. bicarb and extracted twice with ethyl acetate. The combined organic fractions were washed with brine, dried over MgSO₄, filtered, and concentrated in vacuo to give the title compound.

### Intermediate 2. 5-chloro-6-((3,3-dimethyl-2,3-dihydrobenzofuran-4-yl)oxy)pyridin-3-amine

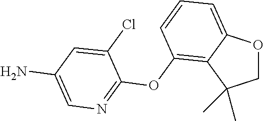 [SMILES: CC1(C)COc2cccc(Oc3ncc(N)cc3Cl)c21]

Intermediate 2 was prepared according to the procedure for Intermediate 1 from 3,3-dimethyl-2,3-dihydrobenzofuran-4-ol (500 mg, 3.04 mmol) and 2,3-dichloro-5-nitropyridine (617 mg, 3.20 mmol).

### Intermediate 3. 6-((4,4-dimethylchroman-5-yl)oxy)pyridin-3-amine

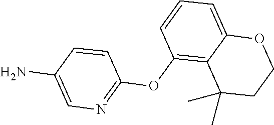 [SMILES: CC1(C)CCOc2cccc(Oc3ccc(N)cn3)c21]

### Step A: 2-((4,4-dimethylchroman-5-yl)oxy)-5-nitropyridine

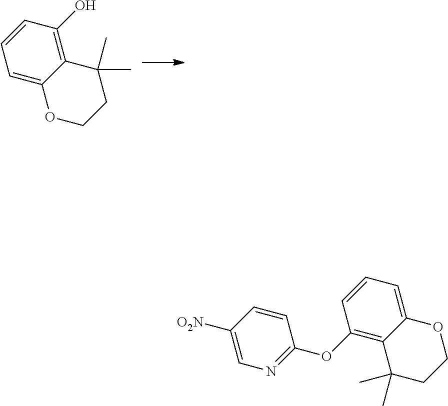 [SMILES: CC1(C)CCOc2cccc(O)c21 ; CC1(C)CCOc2cccc(Oc3ccc([N+](=O)[O-])cn3)c21]

To a mixture of 4,4-dimethylchroman-5-ol (Intermediate 7, 640 mg, 3.59 mmol) in DMF (9 ml)/water (3 ml) was added K₂CO₃ (993 mg, 7.18 mmol) and 2-chloro-5-nitropyridine (598 mg, 3.77 mmol), and the resulting mixture was heated to 60° C. After 16 h, the mixture was added to water and extracted twice with EtOAc. The combined organic fractions were washed with water and brine, dried over MgSO₄, filtered, and concentrated to afford the title compound.

### Step B: 6-((4,4-dimethylchroman-5-yl)oxy)pyridin-3-amine

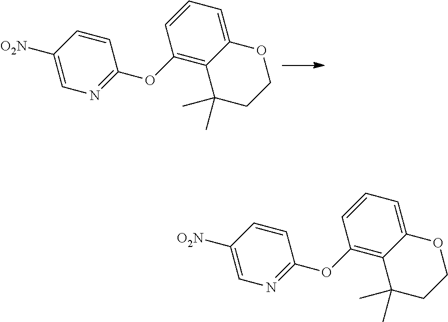 [SMILES: CC1(C)CCOc2cccc(Oc3ccc([N+](=O)[O-])cn3)c21 ; CC1(C)CCOc2cccc(Oc3ccc([N+](=O)[O-])cn3)c21]

To a solution of 2-((4,4-dimethylchroman-5-yl)oxy)-5-nitropyridine (40 mg, 0.133 mmol) in EtOH (5 ml) and water (1 ml) were added iron (29.8 mg, 0.533 mmol) and ammonium chloride (28.5 mg, 0.533 mmol). The mixture was stirred at 80° C. for 2 h, then cooled and filtered. The solvent was evaporated under reduced pressure to give the crude product. The residue was purified by silica gel chromatography (1:1 EA/PE) to give the title compound.

### Intermediate 4. 5-chloro-6-((3,3-dimethyl-2,3-dihydrobenzofuran-4-yl)oxy)-4-methylpyridin-3-amine

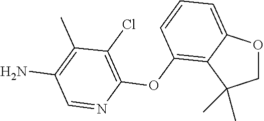 [SMILES: Cc1c(N)cnc(Oc2cccc3c2C(C)(C)CO3)c1Cl]

Intermediate 4 was prepared according to the 2 step procedure for Intermediate 3 from 3,3-dimethyl-2,3-dihydrobenzofuran-4-ol (436 mg. 2.66 mmol) and 2,3-dichloro-4-methyl-5-nitropyridine (500 mg. 2.42 mmol).

### EXAMPLES

### Example 1. 3-(6-(2,3-dimethylphenoxy)pyridin-3-yl)-1,3-dihydro-2H-imidazo[4,5-b]pyridin-2-one

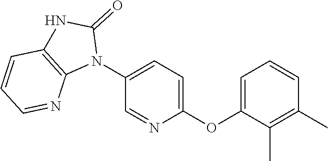 [SMILES: Cc1cccc(Oc2ccc(-n3c(=O)[nH]c4cccnc43)cn2)c1C]

A mixture of 6-(2,3-dimethylphenoxy)pyridin-3-amine (1.9 g, 8.87 mmol), tert-butyl (2-chloropyridin-3-yl)carbamate (2.028 g, 8.87 mmol), chloro[(4,5-bis(diphenylphosphino)-9,9-dimethylxanthene)-2-(2-amino-1,1-biphenyl)]palladium (II) (0.788 g, 0.887 mmol) and Cs₂CO₃ (8.67 g, 26.6 mmol) in DMF (44.3 ml) was stirred at 110° C. After 2 h the mixture was filtered, diluted with water, and extracted with EtOAc three times. The combined organics were washed with water and brine, dried over MgSO₄, filtered and concentrated. Diethyl ether was added to the residue, and the resulting mixture was filtered to afford the title compound as solid. LRMS m/z (M+H): calculated 333.4, observed 333.3. 1H NMR (500 MHz, DMSO-d6) δ 11.48 (s, 1H), 8.36 (d, J=2.5 Hz, 1H), 8.10 (dd, J=8.8, 2.6 Hz. 1H), 7.98-7.87 (m, 1H), 7.41 (d, J=7.7 Hz, 1H), 7.22-7.03 (m, 4H), 6.94 (d, J=7.9 Hz, 1H), 2.30 (s, 3H), 2.05 (s, 3H).

### Example 2. 9-(5-chloro-6-((3,3-dimethyl-2,3-dihydrobenzofuran-4-yl)oxy)pyridin-3-yl)-2-methyl-7,9-dihydro-8H-purin-8-one

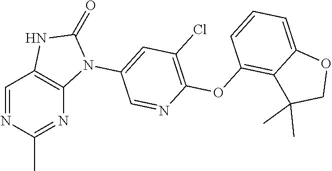 [SMILES: Cc1ncc2[nH]c(=O)n(-c3cnc(Oc4cccc5c4C(C)(C)CO5)c(Cl)c3)c2n1]

### Step A: 3-chloro-2-((3,3-dimethyl-2,3-dihydrobenzofuran-4-yl)oxy)-5-nitropyridine

To a mixture of 3,3-dimethyl-2,3-dihydrobenzofuran-4-ol (500 mg, 3.04 mmol) in DMF (7.61 mL)/water (2.54 mL) was added K₂CO₃ (842 mg, 6.09 mmol) and 2,3-dichloro-5-nitropyridine (617 mg, 3.20 mmol). The resulting mixture was stirred at 60° C. After 16 h, the mixture was added to water and extracted with EtOAc three times. The combined organics were washed twice with water and once with brine, dried over MgSO₄, filtered, and concentrated to a crude residue. Purification by silica chromatography 0-100% EA/hexanes provided the title compound.

### Step B: 5-chloro-6-((3,3-dimethyl-2,3-dihydrobenzofuran-4-yl)oxy)pyridin-3-amine

To a mixture of 3-chloro-2-((3,3-dimethyl-2,3-dihydrobenzofuran-4-yl)oxy)-5-nitropyridine (800 mg, 2.49 mmol) in THF (32.9 mL)/water (10.5 mL)/ethanol (6.49 mL) were added iron (1.393 g, 24.94 mmol), ammonium chloride (667 mg, 12.47 mmol), and acetic acid (0.143 mL, 2.49 mmol). The resulting mixture stirred at rt. After 16 h, the mixture was filtered, and the filtrate was added to saturated aqueous NaHCO₃. The resulting mixture was twice extracted with EtOAc. The combined organics were washed with brine, dried over MgSO₄, filtered and concentrated to give the crude title compound.

### Step C: 9-(5-chloro-6-((3,3-dimethyl-2,3-dihydrobenzofuran-4-yl)oxy)pyridin-3-yl)-2-methyl-7,9-dihydro-8H-purin-8-one

To a flask containing 5-chloro-6-((3,3-dimethyl-2,3-dihydrobenzofuran-4-yl)oxy)pyridin-3-amine (230 mg, 0.791 mmol), tert-butyl (tert-butoxycarbonyl)(4-chloro-2-methylpyrimidin-5-yl)carbamate (340 mg, 0.989 mmol), and tBuXPhos Pd G3 (50 mg, 0.063 mmol) were added DMF (2.6 mL) and sodium tert-butoxide (2 M in THF, 1.19 mL, 2.37 mmol). The resulting mixture was stirred at 120° C. After 8 h, the mixture was filtered and purified by HPLC (10-95% MeCN/H₂O with 0.1% TFA, C18 column) to afford the title compound. LRMS m/z (M+H): calculated 424.9, observed 424.3. 1H NMR (500 MHz, DMSO-d6) δ 8.47-8.34 (m, 2H), 8.25 (s, 1H), 7.24-7.10 (m, 1H), 6.76-6.65 (m, 2H), 4.24 (s, 2H), 2.52 (s, 3H), 1.31 (s, 6H).

### Example 3. 9-(5-chloro-6-((3,3-dimethyl-2,3-dihydrobenzofuran-4-yl)oxy)-4-methylpyridin-3-yl)-2-methyl-7,9-dihydro-8H-purin-8-one

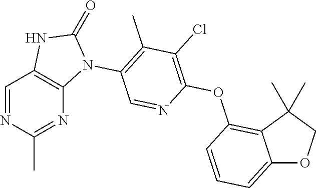 [SMILES: Cc1ncc2[nH]c(=O)n(-c3cnc(Oc4cccc5c4C(C)(C)CO5)c(Cl)c3C)c2n1]

### Step A: 3-chloro-2-((3,3-dimethyl-2,3-dihydrobenzofuran-4-yl)oxy)-4-methyl-5-nitropyridine

To a mixture of 3,3-dimethyl-2,3-dihydrobenzofuran-4-ol (750 mg, 4.57 mmol) in DMF (3.9 mL)/water (1.3 mL) was added K₂CO₃ (1148 mg, 8.31 mmol) and 2,3-dichloro-4-methyl-5-nitropyridine (860 mg, 4.15 mmol). The resulting mixture was stirred at 60° C. After 16 h, the mixture was added to water and extracted with EtOAc three times. The combined organic fractions were washed twice with water and once with brine, dried over MgSO₄, filtered, and concentrated to give a crude residue which was purified by silica chromatography 0-100% EA/hexanes to give the title compound.

### Step B: 5-chloro-6-((3,3-dimethyl-2,3-dihydrobenzofuran-4-yl)oxy)-4-methylpyridin-3-amine

To a mixture of 3-chloro-2-((3,3-dimethyl-2,3-dihydrobenzofuran-4-yl)oxy)-4-methyl-5-nitropyridine (421 mg, 1.26 mmol) in THF (8.3 mL)/water (2.6 mL)/ethanol (1.6 mL) was added iron (702 mg, 12.6 mmol), ammonium chloride (336 mg, 6.29 mmol), and acetic acid (0.072 mL, 1.26 mmol). The resulting mixture was stirred at rt. After 16 h, the mixture was filtered, and the filtrate was added to saturated aqueous NaHCO₃. The resulting mixture was twice extracted with EtOAc. The combined organic fractions were washed with brine, dried over MgSO₄, filtered and concentrated to provide the crude title compound.

### Step C: 9-(5-chloro-6-((3,3-dimethyl-2,3-dihydrobenzofuran-4-yl)oxy)-4-methylpyridin-3-yl)-2-methyl-7,9-dihydro-8H-purin-8-one

To a flask containing 5-chloro-6-((3,3-dimethyl-2,3-dihydrobenzofuran-4-yl)oxy)-4-methylpyridin-3-amine (210 mg, 0.689 mmol), tert-butyl (tert-butoxycarbonyl)(4-chloro-2-methylpyrimidin-5-yl)carbamate (355 mg, 1.03 mmol), and tBuXPhos Pd G3 (109 mg, 0.138 mmol) were added DMF (6.9 mL) and sodium tert-butoxide (2 M in THF, 2.07 mL, 4.14 mmol). The resulting mixture was stirred at 120° C. After 16 h, the mixture was filtered and purified by HPLC (10-95% MeCN/H₂O with 0.1% TFA, C18 column) to afford the title compound. LRMS m/z (M+H): calculated 438.1, observed 438.2. 1H NMR (500 MHz, Chloroform-d) δ 10.30 (s, 1H), 8.32 (s, 1H), 8.04 (s, 1H), 7.17 (t, J=8.1 Hz, 1H), 6.69 (d, J=8.0 Hz, 2H), 4.27 (s, 2H), 2.65 (s, 3H), 2.34 (s, 3H), 1.44 (d, J=13.3 Hz, 6H).

### Biological Assays

A compound of formula (I), (II), or (III) or a pharmaceutically acceptable salt thereof, may be tested using one or both of the biological assays described below to determine its modulatory properties.

### FLIPR Based Tl+ Influx Assay for Characterization of Positive Modulators of Kv3.1

Thallium influx assay was performed following manufacturer instruction with modifications. The major improvement was that the assay was miniaturized in 1536w assay plate to achieve ultrahigh throughput. The cell culture medium used for cell plating was the same medium mentioned above without selection antibiotics. Twenty to twenty four hours prior to the assay day, ARF cells in frozen vials were thawed in 37° C. water bath and washed once in the culture medium. The cell density was adjusted at 8.3×E5/ml and plated in 1536-w PDL coated plates (Aurora. US) at 4 μl/w, using Multidrop Combi (Thermo Fisher Scientific, Waltham, MA USA). Plates were then centrifuged at 300 g for two minutes for cells to achieve uniform settlement. The MicroClime plate lid (Labcyte Inc., San Jose, CA USA) was used to prevent evaporation from the edge wells and minimize the edge effect. Plates were incubated at 37° C., 5% CO₂ overnight.

On the next day, the cell plates were further loaded with 3 μl/w of fluorescence dye solution containing all required components in the assay kit as instructed by the manufacturer protocol and incubated at room temperature for 60 min. Control and test compounds were then added into the cell plate by acoustic liquid transfer Echo (Labcyte Inc., San Jose, CA USA). After 10 min incubation with compounds, the cell plate was transferred to FLIPR Tetra platform (Molecular Devices, Sunnyvale, CA, USA) for recording fluorescence signal. The plate was recorded with excitation wavelength at 475-485 nm and emission wavelength at 510-545 nm. Data acquisition was taken at 1 Hz and completed in 130 seconds. After the first 10 s of recording, 3 μl of stimulus buffer was added into each well by liquid handler equipped in the FLIPR platform. The buffer contains 95 mM of Na₂SO₄ to maintain osmolarity, 7.5 mM of K₂SO₄ to depolarize membrane and activate the voltage gated Kv3.1 or 3.4 channels and 1 mM of Tl₂SO₄ to provide 2 mM Tl+ for Tl sensitive fluorescent signal. The average signal amplitude at 50-60 seconds normalized to the average signal amplitude at 0-10 seconds (ratio of the two means) was used as measure to assess the effect of the test compounds.

### In Vitro Characterization of Positive Modulators of Kv3.1 in Automated Patch Clamp

Automated whole cell patch-clamp was performed on the Qube using stably transfected HEK cell lines overexpressing Kv3.1 channels. Stable cell lines expressing Kv3.2 or Kv3.4 were used for counter-screening. Cells were held at −70 mV. Two depolarizing test pulses to −15 mV (200 ms) and +20 mV (200 ms) were given at a frequency of 0.1 Hz. Kv3.1 current amplitude elicited by test pulses was measured before and after compound addition at steady state. Ten concentrations were tested for each molecule. Raw data from Qube experiments were retrieved from the Qube database and analyzed using the Sophion Analyzer software and DR FIT Spotfire analysis. Apparent compound EC₅₀ values, maximum potentiation, and EC2xIK (effective concentration at which a doubling in K current was observed) were determined using dose response 4 parameter curve fitting.

The compounds disclosed herein have the following KV3.1 EC₅₀ values as shown in Table 1 using the FLIPR and Qube assays, respectively.

TABLE 1
The compounds of the present invention have the following KV3.1
EC₅₀ values in the FLIPR and Qube assays, respectively.

| Ex. No. | FLIPR EC₅₀ (nM) | Qube EC₅₀ (nM) |
|---|---|---|
| 1 | 1630 | 4354 |
| 2 | 145.6 | 212.7 |
| 3 | 31.21 | 67.68 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

What is claimed is:

1. A compound of formula (I), or a pharmaceutically acceptable salt thereof:

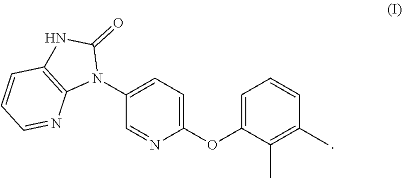 [SMILES: Cc1cccc(Oc2ccc(-n3c(=O)[nH]c4cccnc43)cn2)c1C]

2. A composition comprising a pharmaceutically acceptable carrier and a compound of claim 1, or a pharmaceutically acceptable salt thereof.

3. A method for treating a disease or disorder modulated by Kv3 potassium channels in a subject in need thereof comprising administering to the subject an effective amount of a compound of claim 1, or a pharmaceutically acceptable salt thereof.

4. The method of claim 3, wherein the disease or disorder is a neurological or psychiatric disorder selected from epilepsy, schizophrenia, schizophreniform disorder, schizoaffective disorder, cognitive impairment associated with schizophrenia (CIAS), autism spectrum disorder, bipolar disorder, attention deficit hyperactivity disorder (ADHD), anxiety-related disorder, depression, cognitive dysfunction, Alzheimer's disease, fragile X syndrome, chronic pain, hearing loss, sleep disorder, sleep disruption, Huntington's disease, L-dopa-induced dyskinesia, obsessive-compulsive disorder, and Tourette's syndrome.

5. (canceled)

6. A compound of formula (II), or a pharmaceutically acceptable salt thereof:

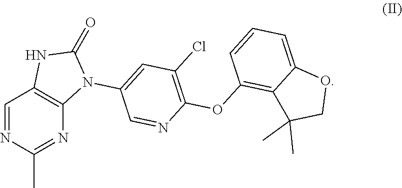 [SMILES: Cc1ncc2[nH]c(=O)n(-c3cnc(Oc4cccc5c4C(C)(C)CO5)c(Cl)c3)c2n1]

7. A composition comprising a pharmaceutically acceptable carrier and a compound of claim 6, or a pharmaceutically acceptable salt thereof.

8. A method for treating a disease or disorder modulated by Kv3 potassium channels in a subject in need thereof comprising administering to the subject an effective amount of a compound of claim 6, or a pharmaceutically acceptable salt thereof.

9. A method for treating a disease or disorder modulated by Kv3.1 potassium channels in a subject in need thereof comprising administering to the subject an effective amount of a compound of claim 6, or a pharmaceutically acceptable salt thereof.

10. The method of claim 9, wherein the disease or disorder is a neurological or psychiatric disorder selected from epilepsy, schizophrenia, schizophreniform disorder, schizoaffective disorder, cognitive impairment associated with schizophrenia (CIAS), autism spectrum disorder, bipolar disorder, attention deficit hyperactivity disorder (ADHD), anxiety-related disorder, depression, cognitive dysfunction, Alzheimer's disease, fragile X syndrome, chronic pain, hearing loss, sleep disorder, sleep disruption, Huntington's disease, L-dopa-induced dyskinesia, obsessive-compulsive disorder, and Tourette's syndrome.

11. (canceled)

12. A compound of formula (III), or a pharmaceutically acceptable salt thereof:

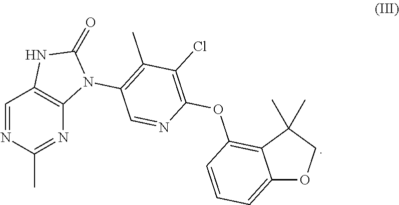 [SMILES: Cc1ncc2[nH]c(=O)n(-c3cnc(Oc4cccc5c4C(C)(C)CO5)c(Cl)c3C)c2n1]

13. A composition comprising a pharmaceutically acceptable carrier and a compound of claim 12, or a pharmaceutically acceptable salt thereof.

14. A method for treating a disease or disorder modulated by Kv3 potassium channels in a subject in need thereof comprising administering to the subject an effective amount of a compound of claim 12, or a pharmaceutically acceptable salt thereof.

15. A method for treating a disease or disorder modulated by Kv3.1 potassium channels in a subject in need thereof comprising administering to the subject an effective amount of a compound of claim 12, or a pharmaceutically acceptable salt thereof.

16. The method of claim 15, wherein the disease or disorder is a neurological or psychiatric disorder selected from epilepsy, schizophrenia, schizophreniform disorder, schizoaffective disorder, cognitive impairment associated with schizophrenia (CIAS), autism spectrum disorder, bipolar disorder, attention deficit hyperactivity disorder (ADHD), anxiety-related disorder, depression, cognitive dysfunction, Alzheimer's disease, fragile X syndrome, chronic pain, hearing loss, sleep disorder, sleep disruption, Huntington's disease, L-dopa-induced dyskinesia, obsessive-compulsive disorder, and Tourette's syndrome.

17. (canceled)
